(19) United States
(12) Patent Application Publication
(10) Pub. No.: US 2025/0011390 A1
(43) Pub. Date: Jan. 9, 2025

(54) Single-Chain Immune Modulator (SCIM)

(71) Applicant: Texas Tech University System, Lubbock, TX (US)

(72) Inventors: Roger B. Sutton, Lubbock, TX (US); Matthew Hirsch, Chapel Hill, NC (US)

(21) Appl. No.: 18/712,507

(22) PCT Filed: Dec. 2, 2022
(86) PCT No.: PCT/US2022/080800

§ 371 Date: May 22, 2024

Related U.S. Application Data
(60) Provisional application No. 63/285,167, filed on Dec. 2, 2021.

(51) Int. Cl. C07K 14/74; A61K 38/00; A61P 27/02; C12N 15/86

(52) U.S. Cl. CPC C07K 14/70539; A61P 27/02; C12N 15/86; A61K 38/00; C07K 2319/00; C12N 2750/14143

(57) ABSTRACT

Provided herein is a fusion protein, expression vector, nucleic acid, or method of making and using a first HLA-G monomer and a second HLA-G monomer connected by a linker peptide expressed as a single chain, and optionally, comprising a providence virus globulin domain to prevent or reduce neovascularization.

### CROSS-REFERENCE TO RELATED APPLICATIONS

This application claims priority to U.S. Provisional Application Ser. No. 63/289,789, filed Dec. 15, 2021, the entire contents of which are incorporated herein by reference.

### TECHNICAL FIELD OF THE INVENTION

The present invention relates in general to the field of immune modulators, and more particularly, to a fusion protein that forms an active HLA-G dimer without the need for disulfide bonding between separate monomers.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ST.26 format via EFS-Web and is hereby incorporated by reference in its entirety. Said ST.26 copy, created on Dec. 2, 2022, is named TECH2166WO_SeqList.xml and is 21, kilo bytes in size.

### BACKGROUND OF THE INVENTION

Without limiting the scope of the invention, its background is described in connection with Human Leukocyte Antigen-G (HLA-G) Histocompatibility Antigen, Class I.

HLA-G is a paralogue of the classic HLA class I proteins and is a transmembrane heterodimer protein that includes, like classic Class I proteins, a heavy chain in association with a β2-microglobulin light chain (known as the HLA-G1 isoform) or a soluble heterodimer (known as the HLA-G5 isoform). The heavy chain of HLA-G includes three globular domains known as the α1, α2, and α3 domains. However, HLA-G molecules are also able to dimerize through the formation of disulfide bonds by reactive cysteine residues between HLA-G monomers. The cysteine residues at position 42 (Cys42-Cys42 bond) and position 147 (Cys147-Cys147 bond) from the HLA-G heavy chain are able to form disulfide bonds.

The dimerization of HLA-G allows receptor binding at higher efficiency and slower dissociation rates than monomeric HLA-G. These interactions are known to be mediated by direct binding of both soluble and membrane bound HLA-G to inhibitory receptors. For example, the binding of HLA-G to the immunoglobulin-like transcript (ILT) receptor 2 on natural killer (NK) cells and T-lymphocytes leads to their inhibition. The HLA-G molecule is also known to inhibit cytotoxic activity of immune cells and to contribute to fetal-maternal tolerance. The HLA-G inhibitory effects may occur through cell-to-cell contact, cell-to-cell dependent uptake of HLA-G or through the release of soluble forms of HLA-G or membrane bound HLA-G shedding. HLA-G is produced as a monomer and binds to another monomer of HLA-G with reduced cysteine residues in order to form the fully active dimer following β-2 microglobulin association.

However, a need remains for improved methods of making fusion proteins that can be used in a variety of settings.

### SUMMARY OF THE INVENTION

As embodied and broadly described herein, an aspect of the present disclosure relates to a fusion protein comprising: a first HLA-G monomer and a second HLA-G monomer connected by a linker peptide expressed as a single chain. In one aspect, the linker peptide is selected from GG, KG, GK, GSG or GGG. In one aspect, the HLA-G is selected from HLA-G1, HLA-G2, HLA-G3, HLA-G4, HLA-G5, HLA-G6, or HLA-G7 or heteromonomers {G1,G1} {G1,G2} {G1,G3} {G1,G4} {G1,G5} {G1,G6} {G1,G7} {G2,G1}{G2,G2} {G2,G3} {G2,G4} {G2,G5} {G2,G6} {G2,G7} {G3,G1} {G3,G2} {G3,G3} {G3,G4} {G3,G5}{G3,G6} {G3,G7} {G4,G1} {G4,G2} {G4,G3} {G4,G4} {G4,G5} {G4,G6} {G4,G7} {G5,G1} {G5,G2}{G5,G3} {G5,G4} {G5,G5} {G5,G6} {G5,G7} {G6,G1} {G6,G2} {G6,G3} {G6,G4} {G6,G5} {G6,G6}{G6,G7} {G7,G1} {G7,G2} {G7,G3} {G7,G4} {G7,G5} {G7,G6} {G7,G7}. In another aspect, the fusion protein further comprises a providence virus globulin domain at an amino- or carboxy-terminus of the fusion protein. In another aspect, the nucleic acid that encodes the HLA-G has 75, 80, 98, 90, 95, 96, 97, 98, 99% or 100% sequence identity with the amino acid sequence of SEQ ID NO: 1 or 2. In another aspect, the HLA-G has amino acid sequence of SEQ ID NO: 3 or 4. In another aspect, the fusion protein is expressed by an expression vector in a bacteria, fungal, yeast, insect, plant, or mammalian cell. In another aspect, the fusion protein is expressed by a vector, RNA, or DNA delivered to a cell by a delivery vehicle or using a delivery method. In another aspect, the fusion protein is a soluble protein. In another aspect, the fusion protein has 75, 80, 98, 90, 95, 96, 97, 98, 99% or 100% sequence identity with the amino acid sequence of SEQ ID NOS: 6 or 8.

As embodied and broadly described herein, an aspect of the present disclosure relates to a method of making a fusion protein comprising: providing a nucleic acid vector that encodes a first HLA-G monomer, a linker sequence, and a second HLA-G monomer, wherein the fusion protein encoded by the nucleic acid forms a single-chain immune modulator protein that is an HLA-G dimer, wherein a transcript from the nucleic acid can be translated into the fusion protein. In one aspect, the linker peptide is selected from GG, KG, GK, GSG or GGG. In one aspect, the HLA-G is selected from HLA-G1, HLA-G2, HLA-G3, HLA-G4, HLA-G5, HLA-G6, or HLA-G7 or heteromonomers {G1,G1} {G1,G2} {G1,G3} {G1,G4}{G1,G5} {G1,G6} {G1,G7} {G2,G1} {G2,G2} {G2,G3} {G2,G4} {G2,G5} {G2,G6} {G2,G7} {G3,G1}{G3,G2} {G3,G3} {G3,G4} {G3,G5} {G3,G6} {G3,G7} {G4,G1} {G4,G2} {G4,G3} {G4,G4} {G4,G5}{G4,G6} {G4,G7} {G5,G1} {G5,G2} {G5,G3} {G5,G4} {G5,G5} {G5,G6} {G5,G7} {G6,G1} {G6,G2}{G6,G3} {G6,G4} {G6,G5} {G6,G6} {G6,G7} {G7,G1} {G7,G2} {G7,G3} {G7,G4} {G7,G5} {G7,G6}{G7,G7}. In another aspect, the fusion protein further comprises a providence virus globulin domain at an amino- or carboxy-terminus of the fusion protein. In another aspect, the nucleic acid that encodes the HLA-G has 75, 80, 98, 90, 95, 96, 97, 98, 99% or 100% sequence identity with the amino acid sequence of SEQ ID NO: 1 or 2. In another aspect, the HLA-G has 75, 80, 98, 90, 95, 96, 97, 98, 99% or 100% sequence identity with the amino acid sequence of amino acid sequence of SEQ ID NO: 3 or 4. In another aspect, the fusion protein is expressed by an expression vector in a bacteria, fungal, yeast, insect, plant, or mammalian cell. In another aspect, the fusion protein is expressed by a vector, RNA, or DNA delivered to a cell by a delivery vehicle or using a delivery method. In another aspect, the fusion protein is a soluble protein. In another aspect, the fusion protein has 75, 80, 98, 90, 95, 96, 97, 98, 99% or 100% sequence identity with the amino acid sequence of SEQ ID NOS: 6 or 8.

As embodied and broadly described herein, an aspect of the present disclosure relates to a single-chain immune modulator protein comprising: a nucleic acid encoding a first HLA-G monomer, a linker sequence, and a second HLA-G monomer, wherein the fusion protein encoded by the nucleic acid forms a single-chain immune modulator protein that resembles or mimics an HLA-G dimer. In one aspect, the HLA-G is selected from HLA-G1, HLA-G2, HLA-G3, HLA-G4, HLA-G5, HLA-G6, or HLA-G7 or heteromonomers {G1,G1} {G1,G2} {G1,G3} {G1,G4} {G1,G5} {G1,G6} {G1,G7} {G2,G1} {G2,G2}{G2,G3} {G2,G4} {G2,G5} {G2,G6} {G2,G7} {G3,G1} {G3,G2} {G3,G3} {G3,G4} {G3,G5} {G3,G6}{G3,G7} {G4,G1} {G4,G2} {G4,G3} {G4,G4} {G4,G5} {G4,G6} {G4,G7} {G5,G1} {G5,G2} {G5,G3}{G5,G4} {G5,G5} {G5,G6} {G5,G7} {G6,G1} {G6,G2} {G6,G3} {G6,G4} {G6,G5} {G6,G6} {G6,G7}{G7,G1} {G7,G2} {G7,G3} {G7,G4} {G7,G5} {G7,G6} {G7,G7}. In another aspect, the nucleic acid further comprises a providence virus globulin domain at an amino- or carboxy-terminus of the fusion protein, or a humanized providence virus globulin domain. In another aspect, the nucleic acid that encodes the HLA-G has SEQ ID NO:1 or 2. In another aspect, the HLA-G has amino acid sequence of SEQ ID NO:3 or 4. In another aspect, the fusion protein is expressed by an expression vector in a bacteria, fungal, yeast, insect, plant, or mammalian cell. In another aspect, the fusion protein is expressed by a vector, RNA, or DNA delivered to a cell by a delivery vehicle or using a delivery method. In another aspect, the nucleic acid expresses a fusion protein that is a soluble protein. In another aspect, the fusion protein has 75, 80, 98, 90, 95, 96, 97, 98, 99% or 100% sequence identity with the nucleic acid sequence of SEQ ID NOS: 5 or 7.

As embodied and broadly described herein, an aspect of the present disclosure relates to a method of preventing neovascularization comprising: providing an animal in need for the prevention of neovascularization with an effective amount of a fusion protein comprising a first HLA-G monomer and a second HLA-G monomer connected by a linker peptide expressed as a single chain. In one aspect, the linker peptide is selected from GG, KG, GK, GSG or GGG. In one aspect, the HLA-G is selected from HLA-G1, HLA-G2, HLA-G3, HLA-G4, HLA-G5, HLA-G6, or HLA-G7 or heteromonomers {G1,G1}{G1,G2} {G1,G3} {G1,G4} {G1,G5} {G1,G6} {G1,G7} {G2,G1} {G2,G2} {G2,G3} {G2,G4} {G2,G5}{G2,G6} {G2,G7} {G3,G1} {G3,G2} {G3,G3} {G3,G4} {G3,G5} {G3,G6} {G3,G7} {G4,G1} {G4,G2}{G4,G3} {G4,G4} {G4,G5} {G4,G6} {G4,G7} {G5,G1} {G5,G2} {G5,G3} {G5,G4} {G5,G5} {G5,G6}{G5,G7} {G6,G1} {G6,G2} {G6,G3} {G6,G4} {G6,G5} {G6,G6} {G6,G7} {G7,G1} {G7,G2} {G7,G3}{G7,G4} {G7,G5} {G7,G6} {G7,G7}. In another aspect, the fusion protein further comprises a providence virus globulin domain at an amino- or carboxy-terminus of the fusion protein. In another aspect, the nucleic acid that encodes the HLA-G has SEQ ID NO:1 or 2. In another aspect, the HLA-G has amino acid sequence of SEQ ID NO:3 or 4. In another aspect, the protein is expressed by an expression vector. In another aspect, the fusion protein is expressed by an expression vector in a bacteria, fungal, yeast, insect, plant, or mammalian cell. In another aspect, the fusion protein is expressed by a vector, RNA, or DNA delivered to a cell by a delivery vehicle or using a delivery method. In another aspect, the expression vector is a viral, bacteria, fungal, yeast, insect, plant, or mammalian expression vector. In another aspect, the fusion protein is a soluble protein. In another aspect, the fusion protein has 75, 80, 98, 90, 95, 96, 97, 98, 99% or 100% sequence identity with the amino acid sequence of SEQ ID NOS: 6 or 8.

As embodied and broadly described herein, an aspect of the present disclosure relates to a method of preventing or reducing neovascularization of the cornea comprising: providing an animal in need for the prevention of neovascularization of the cornea with an effective amount of a fusion protein comprising a first HLA-G monomer and a second HLA-G monomer connected by a linker peptide expressed as a single chain to prevent or reduce the neovascularization of the cornea.

### BRIEF DESCRIPTION OF THE DRAWINGS

For a more complete understanding of the features and advantages of the present invention, reference is now made to the detailed description of the invention along with the accompanying figures and in which:

FIG. 1A shows an example of a three-dimensional model of the single chain immunomodulator (scIM) of the present invention.

FIG. 1B shows a Western Blot of the expressed scIM-2 protein vs. the wild-type monomeric HLA-G1 proteins.

FIG. 2 shows an annotated sequence alignment of the scIM-1 (SEQ ID NO: 6) and scIM-2 (SEQ ID NO: 8, beginning at pos. 4) when compared to HLA-G (a segment of SEQ ID NO: 3 or 4) and 2qqp (SEQ ID NO: 9).

FIG. 3 is an example of a self-complementary adeno associated virus (AAV) vector map for the scIM of the present invention, in this example the insert is scIM-2.

FIG. 4 is a graph that shows the vascular score for the prevention of corneal neovascularization post-NaOH corneal burn when treated with phosphate buffered saline (PBS), AAV-green fluorescence protein (GFP), AAV-scIM-1, or AAV-scIM-2, all injected subconjunctivally.

FIG. 5A shows that scAAV8-scIMs (scIM-1 and scIM-2) prevent NaOH corneal burn induced neovascularization, the controls are animals treated with PBS and AAV8-GFP.

FIG. 5B are histological cross-section of corneas treated with AAV8-GFP and AAV8-scIM1, demonstrating decreased burn-induced cellular infiltration of the cornea.

FIG. 6 shows the mean rejection index scores after full-thickness high-risk corneal transplantation of donor corneas incubated in AAV8-scIM1, AAV8-scIM2, or vehicle in rabbits. Rejection index (RI) score, a scale of 0 to 12, is based on the combined scores of 3 corneal graft indicators: clarity, edema, and neovascularization, each of which is scored on a scale of 0 to 4, with 0 being normal and 4 being of highest severity. A cornea with an RI score of 6 is considered rejected (dotted horizontal line). Corneas incubated with saline developed a mean RI of 6 between 17 and 21 days after surgery. Two of 6 eyes incubated in AAV8-scIM2 had RI scores of 6 by day 29 after surgery. The remaining corneas incubated in AA8-scIM2 and all corneas incubated in AAV8-scIM2 did not reject through day 42 after surgery.

FIG. 7 shows individual central corneal thickness measurements after full-thickness high-risk corneal transplantation of donor corneas incubated in AAV8-scIM1, AAV8-scIM2, or vehicle in rabbits. A higher corneal thickness suggests corneal decompensation with inflammation. All but 2 corneas incubated with AAV8-scIM2 and all corneas incubated in AAV8-scIM1 had central corneal thickness measurements that returned to normal by day 21-24 days after surgery.

FIG. 8 shows photographs after full-thickness corneal transplantation of donor corneas incubated in AAV8-scIM1 or vehicle in rabbits. Healthy corneal grafts were incubated in vehicle or AAV8-scIM1 for 15 min prior to surgical engraftment into a high-risk corneal recipient bed. Representative photographs depict complete corneal graft rejection (PBS) or tolerance (AAV8-scIM1) at Day 38 post-engraftment.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Although produced as a monomer, the disulfide-linked dimer form of HLA-G is enhanced for receptor affinity, eliciting intracellular signaling, and thus overall function. Consequently, AAV-delivered HLA-G must express in the target cells and bind to another monomer of HLA-G, with reduced reactive cysteine residues, to form a fully active dimer following beta-2 microglobulin (B2M) association. A more efficient mechanism would be to deliver active HLA-G dimers as a single gene that can produce a monomeric polypeptide chain with similar bioactivity in a manner independent of B2M. Single gene delivery would bypass the slow kinetics of disulfide formation, and place adequate amounts of active proteins in the proper tissues. Furthermore, using in silico modeling, this invention alleviates the requirement for the association of HLA-based molecules with beta-2 microglobulin (B2M), another rate limiting step for HLA-G function. The present invention is a single chain immune modulator (scIMs) capable of functioning as dimeric protein complexes as a single polypeptide.

As used herein, the term “delivery vehicle” or “delivery method” refers to any vehicle that allows the transfer of a nucleic acid (e.g., RNA, DNA), protein, and/or a vector into a cell. Non-limiting examples of delivery vehicles or methods for delivery include: chemical based delivery vehicle (e.g., calcium phosphate, cationic polymers, cationic liposomes, cyclodextrin), protein-based or peptide-based delivery vehicles, lipid-based delivery vehicles, nanoparticle-based delivery vehicles, non-chemical-based delivery vehicles (e.g., electroporation, sonoporation, optical transfection, transformation), particle-based delivery vehicles (e.g., gene gun, magnetofection, particle bombardment, cell-penetrating peptides), a viral delivery, a viral-like particle, or a virus scaffold.

As used herein, the term “fusion protein” refers to a hybrid protein, that includes portions of two or more different polypeptides, or fragments thereof, resulting from the expression of a polynucleotide that encodes at least a portion of each of the two polypeptides.

As used herein, the term “gene” refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “homology,” refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence.

As used herein, the term “host cell” refers to cells that have been engineered to contain nucleic acid segments or altered segments, whether archeal, prokaryotic, or eukaryotic. Thus, engineered, or recombinant cells, are distinguishable from naturally occurring cells that do not contain recombinantly introduced nucleic acids.

As used herein the term “Major Histocompatibility Complex” or “MHC” refers to the proteins of the Major Histocompatibility Complex, which are a set of gene loci the encode major histocompatibility antigens. The term “HLA” as used herein will be understood to refer to Human Leukocyte Antigens, which is defined as the MHC found in humans. As used herein, “HLA” is the human form of “MHC”.

As used herein the terms “protein”, “polypeptide” or “peptide” refer to compounds comprising amino acids joined via peptide bonds and are used interchangeably.

As used herein, the term “transformation,” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome.

As used herein, the term “transfection” refers to the introduction of foreign DNA into eukaryotic cells. Transfection may be accomplished by a variety of methods known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell that has stably integrated foreign DNA into the genomic DNA. The term also encompasses cells that transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “treatment” refers to both therapeutic treatment and prophylactic or preventative measures. Those in need of treatment include those already with the disorder as well as those in which the disorder is to be prevented. For example, the present invention can be used to treat or prevent inflammatory, diseases, neovascular diseases, any organ/tissue and cell rejection.

As used herein, the term “vector” refers to nucleic acid molecules that transfer RNA or DNA segment(s) into a cell. The term “vehicle” is sometimes used interchangeably with “vector.” The term “vector” as used herein also includes RNA, DNA, and/or expression vectors in reference to a recombinant RNA or DNA molecule containing a desired coding sequence and appropriate nucleic acid sequences necessary for the expression of the fusion proteins of the present invention in a particular host organism. Nucleic acid sequences necessary for expression in eukaryotes usually include promoters, enhancers, and termination and polyadenylation signals. Thus, a vector can include viral and non-viral delivery formats using in protein, RNA, or DNA delivery vehicles. The vehicle can be adapted for any route of administration and may also be used ex vivo (stem cell, organ, or cell transplants). For example, the present invention can include cells lines with scIM integrated into the chromosomes, universal stem cell donors (for all indications), and transient transfection.

The present invention can be used in transplantation of organs and tissues a treatment option for many patients with chronic diseases. However, immune rejection of transplanted organs is a major obstacle that requires a lifetime of anti-rejection drugs or repeated transplantation. Dimeric HLA-G inhibits the cytolytic function of natural killer cells (NK cells); hence exogenously expressed HLA-G could be used as a constitutive immune modulator in place of a lifetime of anti-rejection drugs.

The present invention is an HLA-G is an MHC class I molecule with immunological characteristics to prevent rejection in transplanted patients. Presently, patients are treated with immunosuppressants such as Tacrolimus or cyclosporin for life. If the transplant is rejected, additional surgeries are required to re-transplant the organ.

Unlike the HLA-G protein, scIMs (single-chain immune modulators) are monomeric proteins. As such, transfection of transplanted patients with AAV-scIMs requires significantly less virus since the kinetically-slowest steps of HLA-G dimerization are eliminated.

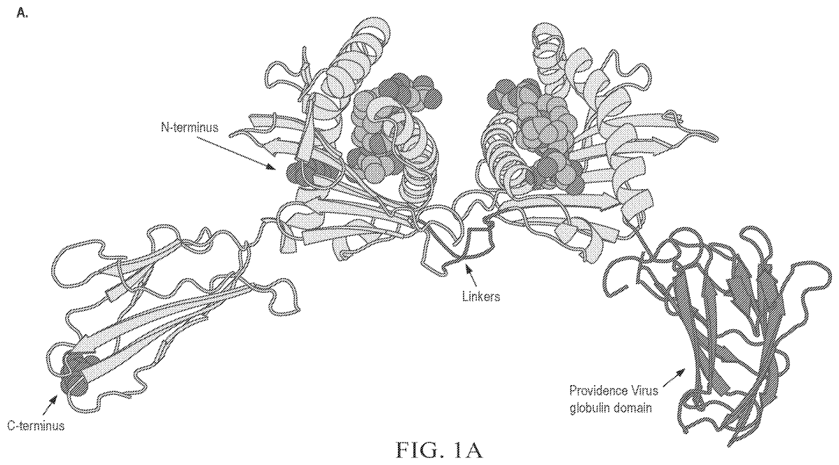

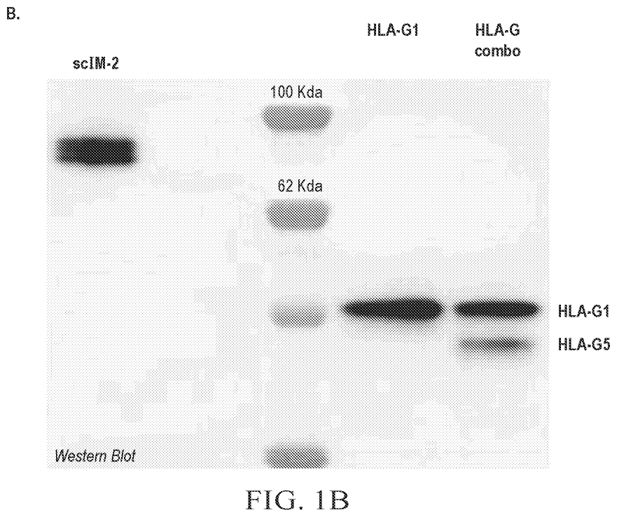

In one example, the present invention includes inserting the scIMs in an Adeno-associated virus as the delivery vector, this bypasses the slow kinetics of disulfide bond formation. Additionally, transfection transplanted patients with AAV-scIM would require significantly less virus since the kinetically-slowest steps of HLA-G dimerization are eliminated. The scIMs gene has been successfully built and produces a soluble protein in cell culture, a representation of the structure is shown in FIG. 1A, while the western blot of the expressed scIM-2 protein compared to the wild-type monomeric HLA-G1 proteins and HLA-G1-HLA-G5 combo are shown in FIG. 1B.

- HLA-G, Accession No. NM_002127
SEQ ID NO: 1
AGTGTGGTAC TTTGTCTTGA GGAGATGTCC TGGACTCACA CGGAAACTTA GGGCTACGGA
ATGAAGTTCT CACTCCCATT AGGTGACAGG TTTTTAGAGA AGCCAATCAG CGTCGCCGCG
GTCCTGGTTC TAAAGTCCTC GCTCACCCAC CCGGACTCAT TCTCCCCAGA CGCCAAGGAT
GGTGGTCATG GCGCCCCGAA CCCTCTTCCT GCTGCTCTCG GGGGCCCTGA CCCTGACCGA
GACCTGGGCG GGCTCCCACT CCATGAGGTA TTTCAGCGCC GCCGTGTCCC GGCCCGGCCG
CGGGGAGCCC CGCTTCATCG CCATGGGCTA CGTGGACGAC ACGCAGTTCG TGCGGTTCGA
CAGCGACTCG GCGTGTCCGA GGATGGAGCC GCGGGCGCCG TGGGTGGAGC AGGAGGGGCC
GGAGTATTGG GAAGAGGAGA CACGGAACAC CAAGGCCCAC GCACAGACTG ACAGAATGAA
CCTGCAGACC CTGCGCGGCT ACTACAACCA GAGCGAGGCC AGTTCTCACA CCCTCCAGTG
GATGATTGGC TGCGACCTGG GGTCCGACGG ACGCCTCCTC CGCGGGTATG AACAGTATGC
CTACGATGGC AAGGATTACC TCGCCCTGAA CGAGGACCTG CGCTCCTGGA CCGCAGCGGA
CACTGCGGCT CAGATCTCCA AGCGCAAGTG TGAGGCGGCC AATGTGGCTG AACAAAGGAG
AGCCTACCTG GAGGGCACGT GCGTGGAGTG GCTCCACAGA TACCTGGAGA ACGGGAAGGA
GATGCTGCAG CGCGCGGACC CCCCCAAGAC ACACGTGACC CACCACCCTG TCTTTGACTA
TGAGGCCACC CTGAGGTGCT GGGCCCTGGG CTTCTACCCT GCGGAGATCA TACTGACCTG
GCAGCGGGAT GGGGAGGACC AGACCCAGGA CGTGGAGCTC GTGGAGACCA GGCCTGCAGG
GGATGGAACC TTCCAGAAGT GGGCAGCTGT GGTGGTGCCT TCTGGAGAGG AGCAGAGATA
CACGTGCCAT GTGCAGCATG AGGGGCTGCC GGAGCCCCTC ATGCTGAGAT GGAAGCAGTC
TTCCCTGCCC ACCATCCCCA TCATGGGTAT CGTTGCTGGC CTGGTTGTCC TTGCAGCTGT
AGTCACTGGA GCTGCGGTCG CTGCTGTGCT GTGGAGAAAG AAGAGCTCAG ATTGAAAAGG
AGGGAGCTAC TCTCAGGCTG CAATGTGAAA CAGCTGCCCT GTGTGGGACT GAGTGGCAAG
TCCCTTTGTG ACTTCAAGAA CCCTGACTCC TCTTTGTGCA GAGACCAGCC CACCCCTGTG
CCCACCATGA CCCTCTTCCT CATGCTGAAC TGCATTCCTT CCCCAATCAC CTTTCCTGTT
CCAGAAAAGG GGCTGGGATG TCTCCGTCTC TGTCTCAAAT TTGTGGTCCA CTGAGCTATA
ACTTACTTCT GTATTAAAAT TAGAATCTGA GTATAAATTT ACTTTTTCAA ATTATTTCCA
AGAGAGATTG ATGGGTTAAT TAAAGGAGAA GATTCCTGAA ATTTGAGAGA CAAAATAAAT
GGAAGACATG AGAACTTT
- HLA-G, Accession No. NM_001363567
SEQ ID NO: 2
AGTGTGGTAC TTTGTCTTGA GGAGATGTCC TGGACTCACA CGGAAACTTA GGGCTACGGA
ATGAAGACGC CAAGGATGGT GGTCATGGCG CCCCGAACCC TCTTCCTGCT GCTCTCGGGG
GCCCTGACCC TGACCGAGAC CTGGGCGGGC TCCCACTCCA TGAGGTATTT CAGCGCCGCC
GTGTCCCGGC CCGGCCGCGG GGAGCCCCGC TTCATCGCCA TGGGCTACGT GGACGACACG
CAGTTCGTGC GGTTCGACAG CGACTCGGCG TGTCCGAGGA TGGAGCCGCG GGCGCCGTGG
GTGGAGCAGG AGGGGCCGGA GTATTGGGAA GAGGAGACAC GGAACACCAA GGCCCACGCA
CAGACTGACA GAATGAACCT GCAGACCCTG CGCGGCTACT ACAACCAGAG CGAGGCCAGT
TCTCACACCC TCCAGTGGAT GATTGGCTGC GACCTGGGGT CCGACGGACG CCTCCTCCGC
GGGTATGAAC AGTATGCCTA CGATGGCAAG GATTACCTCG CCCTGAACGA GGACCTGCGC
TCCTGGACCG CAGCGGACAC TGCGGCTCAG ATCTCCAAGC GCAAGTGTGA GGCGGCCAAT
GTGGCTGAAC AAAGGAGAGC CTACCTGGAG GGCACGTGCG TGGAGTGGCT CCACAGATAC
CTGGAGAACG GGAAGGAGAT GCTGCAGCGC GCGGACCCCC CCAAGACACA CGTGACCCAC
CACCCTGTCT TTGACTATGA GGCCACCCTG AGGTGCTGGG CCCTGGGCTT CTACCCTGCG
GAGATCATAC TGACCTGGCA GCGGGATGGG GAGGACCAGA CCCAGGACGT GGAGCTCGTG
GAGACCAGGC CTGCAGGGGA TGGAACCTTC CAGAAGTGGG CAGCTGTGGT GGTGCCTTCT
GGAGAGGAGC AGAGATACAC GTGCCATGTG CAGCATGAGG GGCTGCCGGA GCCCCTCATG
CTGAGATGGA AGCAGTCTTC CCTGCCCACC ATCCCCATCA TGGGTATCGT TGCTGGCCTG
GTTGTCCTTG CAGCTGTAGT CACTGGAGCT GCGGTCGCTG CTGTGCTGTG GAGAAAGAAG
AGCTCAGATT GAAAAGGAGG GAGCTACTCT CAGGCTGCAA TGTGAAACAG CTGCCCTGTG
TGGGACTGAG TGGCAAGTCC CTTTGTGACT TCAAGAACCC TGACTCCTCT TTGTGCAGAG
ACCAGCCCAC CCCTGTGCCC ACCATGACCC TCTTCCTCAT GCTGAACTGC ATTCCTTCCC
CAATCACCTT TCCTGTTCCA GAAAAGGGGC TGGGATGTCT CCGTCTCTGT CTCAAATTTG
TGGTCCACTG AGCTATAACT TACTTCTGTA TTAAAATTAG AATCTGAGTA TAAATTTACT
TTTTCAAATT ATTTCCAAGA GAGATTGATG GGTTAATTAA AGGAGAAGAT TCCTGAAATT
TGAGAGACAA AATAAATGGA AGACATGAGA ACTTT
- HLA-G, Accession No. NP_002118.1
SEQ ID NO: 3
MVVMAPRTLFLLLSGALTLTETWAGSHSMRYFSAAVSRPGRGEPRFIAMGYVDDTQFVRFDSD
SACPRMEPRAPWVEQEGPEYWEEETRNTKAHAQTDRMNLQTLRGYYNQSEASSHTLQWMIGC
DLGSDGRLLRGYEQYAYDGKDYLALNEDLRSWTAADTAAQISKRKCEAANVAEQRRAYLEGT
CVEWLHRYLENGKEMLQRADPPKTHVTHHPVFDYEATLRCWALGFYPAEIILTWQRDGEDQTQ
DVELVETRPAGDGTFQKWAAVVVPSGEEQRYTCHVQHEGLPEPLMLRWKQSSLPTIPIMGIVAG
LVVLAAVVTGAAVAAVLWRKKSSD
- HLA-G, Accession No. NP_001350396
SEQ ID NO: 4
KTPRMVVMAPRTLFLLLSGALTLTETWAGSHSMRYFSAAVSRPGRGEPRFIAMGYVDDTQFVR
FDSDSACPRMEPRAPWVEQEGPEYWEEETRNTKAHAQTDRMNLQTLRGYYNQSEASSHTLQW
MIGCDLGSDGRLLRGYEQYAYDGKDYLALNEDLRSWTAADTAAQISKRKCEAANVEAQRRAY
LEGTCVEWLHRYLENGKEMLQRADPPKTHVTHHPVFDYEATLRCWALGFYPAEIILTWQRDGE
DQTQDVELVETRPAGDGTFQKWAAVVVPSGEEQRYTCHVQHEGLPEPLMLRWKQSSLPTIPIM
GIVAGLVVLAAVVTGAAVAAVLWRKKSSD
scIM1:
- nucleic acid sequence
SEQ ID NO: 5
ATGAGGTACTTCTCCGCCGCCGTGTCTCGGCCAGGAAGAGGAGAGCCACGGTTCATCGCTAT
GGGCTATGTGGACGATACCCAGTTCGTGAGGTTTGACAGCGATTCCGCCGGACCAGAGCGG
TTCTACGAGAAGCCTCGGATGGAGCCAAGAGCCCCCTGGGTGGAGCAGGAGGGACCTGAGT
ATTGGGAGGAGGAGACCAGGAACACAAAGGCCCACGCCCAGACAGACAGGATGAACCTGC
AGACCCTGCGCGGCTACTATAATCAGTCTGAGGCCAGCTCCCACACCCTGCAGTGGATGATC
GGATGCGACCTGGGAAGCGATGGCAGGCTGCTGAGGGGATACGAGCAGTACGCCTATGACG
GCAAGGATTATCTGGCCCTGAATGAGGACCTGCGGTCCTGGACAGCAGCAGATACCGCAGC
CCAGATCTCTAAGAGAAAGTGCGAGGCAGCAAACGTGGCAGAGCAGAGGAGAGCATACCT
GGAGGGAACCTGCGTGGAGTGGCTGCACCGGTATCTGGAGAATGGACCACCTGCAGAGAGA
TTCAGCGCCGGATCCATGACAGTGTCTGCCAGCAACGCCATCTTTCAGCCCTCCAATACCGT
GGCCCGCATCGTGTGGTCTATCACACCTCTGCCAGTGGCAACCGTGGCCCTGACCACAGGAA
CCGGCGGCACAAACAATACCTCTGGCAAGTTCTTTAGCGTGGAGATCGACGGCAACGTGAA
TAGCGTGTGGACCTTCACAGCACCAGCATCCATCCTGGCAGAGGGAGAGCCCTTCGCCGAG
GAGGGCGACACCACATCCTTTTCTATGACCACAATCACAGCCGATACCGTGGTGTACTCCGT
GTCTAGCTCCCTGACAGGCTCTAGCGTGATCGTGCGGGGAGTGACCAAGGGATCTGGCGTG
AGCATCACACCAGTGACCGTGGGAATCGACACAGGAGGAGGAATGAGGTACTTCAGCGCCG
CCGTGTCCCGGCCTGGAAGAGGAGAGCCACGCTTTATCGCTATGGGCTACGTCGATGACACC
CAGTTCGTGCGGTTTGATTCTGGCAGAATCTTTAAGCAGGTGCCAGGAAGCGCCCCTAGGAT
GGAGCCTCGCGCCCCCTGGGTCGAACAGGAGGGACCAGAGTACTGGGAGGAGGAGACAAG
AAACACCAAAGCTCACGCCCAGACAGATAGAATGAACCTGCAGACCCTGAGAGGCTACTAT
AATCAGTCCGAGGCCTCCTCTCATACTCTGCAGTGGATGATCGGCTGTGACCTGGGCTCCGA
CGGCAGGCTGCTGAGAGGCTATGAACAGTATGCTTACGATGGCAAGGACTATCTGGCCCTG
AATGAAGACCTGAGGAGCTGGACCGCCGCAGATACCGCAGCACAGATCTCCAAGAGGAAAT
GTGAAGCCGCCAACGTGGCCGAGCAGAGGAGGGCATACCTGGAGGGAACATGCGTGGAAT
GGCTGCATAGGTATCTGGAGAATGGCAAGGAGATGCTGCAGCGCGCCGACCCACCCAAGAC
ACACGTGACCCACCACCCCGTGTTCGATTACGAGGCCACCCTGAGGTGCTGGGCACTGGGCT
TTTATCCTGCCGAGATCATCCTGACATGGCAGAGGGACGGAGAGGATCAGACCCAGGACGT
GGAGCTGGTGGAGACAAGACCTGCCGGCGATGGCACCTTTCAGAAGTGGGCAGCAGTGGTG
GTGCCAAGCGGAGAGGAGCAGAGGTACACCTGTCACGTGCAGCACGAGGGACTGCCAGAG
CCTCTGATGCTGAGGTGGAAGCAGtga
scIM1:
- amino acid sequence
SEQ ID NO: 6
MRYFSAAVSR PGRGEPRFIA MGYVDDTQFV RFDSDSAGPE RFYEKPRMEP RAPWVEQEGP
EYWEEETRNT KAHAQTDRMN LQTLRGYYNQ SEASSHTLQW MIGCDLGSDG RLLRGYEQYA
YDGKDYLALN EDLRSWTAAD TAAQISKRKC EAANVAEQRR AYLEGTCVEW LHRYLENGPP
AERFSAGSMT VSASNAIFQP SNTVARIVWS ITPLPVATVA LTTGTGGTNN TSGKFFSVEI
DGNVNSVWTF TAPASILAEG EPFAEEGDTT SFSMTTITAD TVVYSVSSSL TGSSVIVRGV
TKGSGVSITP VTVGIDTGGG MRYFSAAVSR PGRGEPRFIA MGYVDDTQFV RFDSGRIFKQ
VPGSAPRMEP RAPWVEQEGP EYWEEETRNT KAHAQTDRMN LQTLRGYYNQ SEASSHTLQW
MIGCDLGSDG RLLRGYEQYA YDGKDYLALN EDLRSWTAAD TAAQISKRKC EAANVAEQRR
AYLEGTCVEW LHRYLENGKE MLQRADPPKT HVTHHPVFDY EATLRCWALG FYPAEIILTW
QRDGEDQTQD VELVETRPAG DGTFQKWAAV VVPSGEEQRY TCHVQHEGLP EPLMLRWKQ
scIM2:
- nucleic acid sequence
SEQ ID NO: 7
ATGGTCGTGATGAGGTACTTCTCCGCCGCCGTGAGCCGGCCTGGAAGGGGAGAGCCCAGGT
TCATCGCTATGGGCTACGTGGACGACACCCAGTTCGTGAGATTCGACTCCGACAGCGCCGGA
CCAAAGCCTAGGATGGAGCCACGCGCCCCTTGGGTGGAGCAGGAGGGCCCCGAGTACTGGG
AGGAGGAGACCAGAAACACCAAGGCCCACGCCCAGACCGACAGGATGAACCTGCAGACCC
TGAGAGGCTACTACAACCAGTCCGAGGCCAGCTCCCACACCCTGCAGTGGATGATCGGATG
CGACCTGGGAAGCGACGGCAGGCTGCTGAGAGGCTACGAGCAGTACGCCTACGACGGCAA
GGACTACCTGGCCCTGAACGAGGACCTGCGGTCCTGGACCGCAGCAGACACCGCAGCCCAG
ATCAGCAAGAGGAAGTGCGAGGCAGCAAACGTGGCAGAGCAGAGGAGGGCCTACCTGGAG
GGCACCTGCGTGGAGTGGCTGCACAGGTACCTGGAGAACGGACCTCCAGCAGAGAGATTCT
CCGCCGGAAGCATGACCGTGTCCGCCAGCAACGCCATCTTCCAGCCTTCCAACACCGTGGCC
AGGATCGTGTGGAGCATCACCCCTCTGCCAGTGGCAACCGTGGCCCTGACCACCGGAACCG
GAGGAACCAACAACACCTCCGGCAAGTTCTTCAGCGTGGAGATCGACGGCAACGTGAACTC
CGTGTGGACCTTCACCGCACCTGCAAGCATCCTGGCAGAGGGAGAGCCATTCGCCGAGGAG
GGCGACACCACCTCCTTCAGCATGACCACCATCACCGCCGACACCGTGGTGTACTCCGTGAG
CTCCAGCCTGACCGGCTCCAGCGTGATCGTGCGGGGAGTGACCAAGGGATCCGGCGTGAGC
ATCACCCCAGTGACCGTGGGAATCGACACCGGAGGAGGAATGAGATACTTCTCCGCCGCCG
TGAGCCGGCCGGGAAGGGGAGAGCCTCGCTTCATCGCTATGGGCTATGTCGATGACACCCA
GTTCGTGCGGTTCGACTCCGGCAGGATCGGAAGCGCCCCTAGGATGGAGCCCAGAGCCCCC
TGGGTCGAACAGGAAGGCCCCGAGTATTGGGAGGAGGAGACCAGGAACACCAAGGCACAC
GCACAGACCGACAGAATGAACCTGCAGACCCTGCGCGGATATTATAATCAGTCCGAGGCCT
CCAGCCATACTCTCCAGTGGATGATTGGCTGTGACCTGGGCTCCGATGGCAGGCTGCTCAGG
GGATATGAACAGTATGCTTATGATGGCAAGGATTACCTGGCCCTGAACGAAGACCTGAGGT
CCTGGACTGCCGCAGACACCGCAGCACAGATCAGCAAGAGAAAATGTGAAGCCGCCAACGT
GGCCGAGCAGAGGAGAGCCTACCTGGAGGGAACTTGCGTGGAATGGCTGCACCGGTACCTG
GAGAACGGCAAGGAGATGCTGCAGAGGGCAGACCCTCCTAAGACCCACGTGACCCACCACC
CCGTGTTCGACTACGAGGCCACCCTGAGGTGCTGGGCACTGGGCTTCTACCCAGCCGAGATC
ATCCTGACCTGGCAGAGGGACGGAGAGGACCAGACCCAGGACGTGGAGCTGGTGGAGACC
AGACCAGCAGGCGACGGCACCTTCCAGAAGTGGGCAGCAGTGGTGGTGCCTAGCGGCGAGG
AGCAGCGGTACACCTGCCACGTGCAGCACGAGGGACTGCCTGAGCCTCTGATGCTGAGGTG
GAAGCAGCTCGAGAGGCCTAATAAAGAGCTCAGATGCATCGATCAGAGTGTGTTGGTTTTTT
GTGTGACGCGTAGGAACCCCTAGTGA
scIM2:
- amino acid sequence
SEQ ID NO: 8
MVVMRYFSAAVSRPGRGEPRFIAMGYVDDTQFVRFDSDSAGPKPRMEPRAPWVEQEGPEYWE
EETRNTKAHAQTDRMNLQTLRGYYNQSEASSHTLQWMIGCDLGSDGRLLRGYEQYAYDGKDY
LALNEDLRSWTAADTAAQISKRKCEAANVAEQRRAYLEGTCVEWLHRYLENGPPAERFSAGSM
TVSASNAIFQPSNTVARIVWSITPLPVATVALTTGTGGTNNTSGKFFSVEIDGNVNSVWTFTAPAS
ILAEGEPFAEEGDTTSFSMTTITADTVVYSVSSSLTGSSVIVRGVTKGSGVSITPVTVGIDTGGGM
RYFSAAVSRPGRGEPRFIAMGYVDDTQFVRFDSGRIGSAPRMEPRAPWVEQEGPEYWEEETRNT
KAHAQTDRMNLQTLRGYYNQSEASSHTLQWMIGCDLGSDGRLLRGYEQYAYDGKDYLALNE
DLRSWTAADTAAQISKRKCEAANVAEQRRAYLEGTCVEWLHRYLENGKEMLQRADPPKTHVT
HHPVFDYEATLRCWALGFYPAEIILTWQRDGEDQTQDVELVETRPAGDGTFQKWAAVVVPSGE
EQRYTCHVQHEGLPEPLMLRWKQLERPNKELRCIDQSVLVFCVTRRNP*

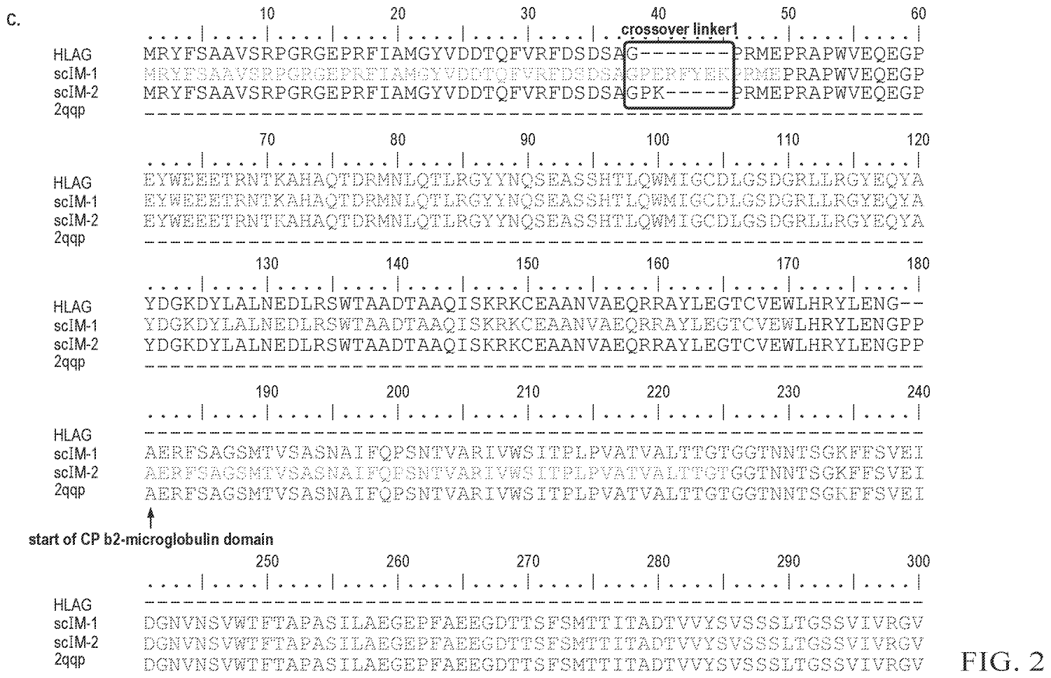

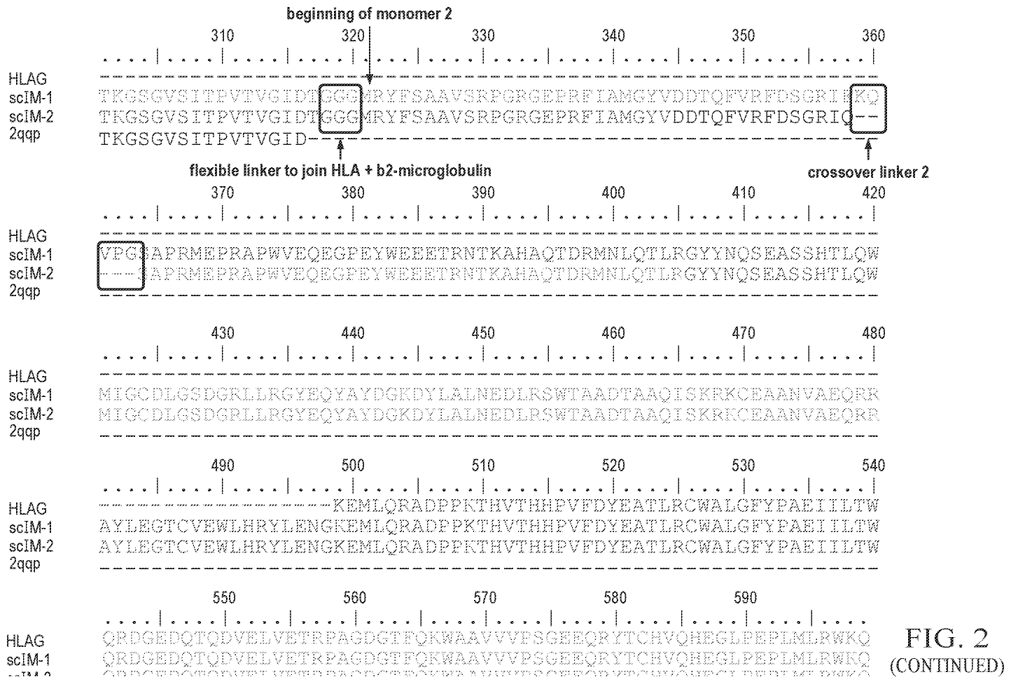

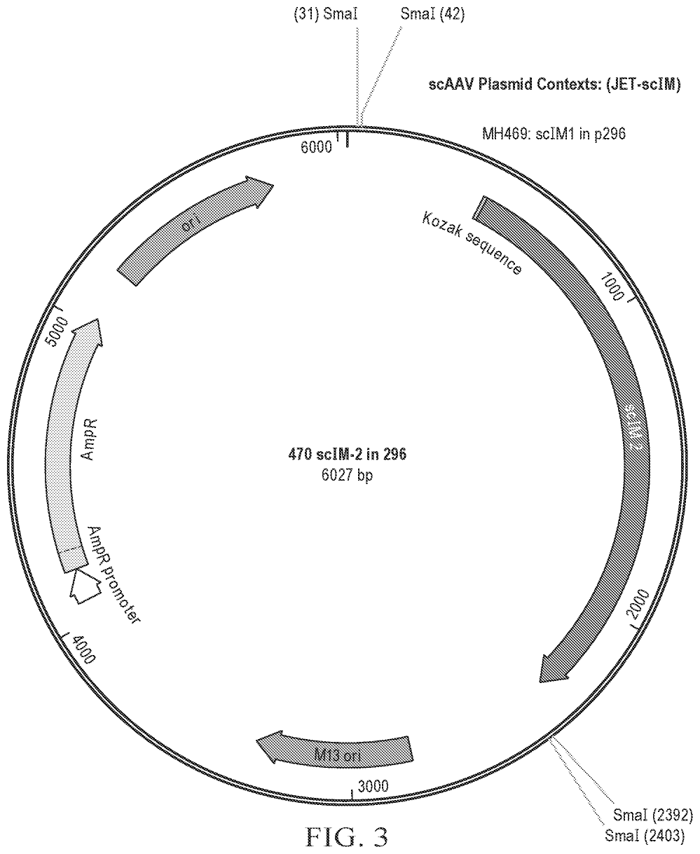

FIG. 2 shows an annotated sequence alignment of the scIM-1 (SEQ ID NO: 6) and scIM-2 (SEQ ID NO: 8, beginning at pos. 4) when compared to HLA-G (a segment of SEQ ID NO: 3 or 4) and 2qqp (SEQ ID NO: 9). FIG. 3 is an example of a self-complementary adeno associated virus (AAV) vector map for the scIM of the present invention, in this example the insert is scIM-2.

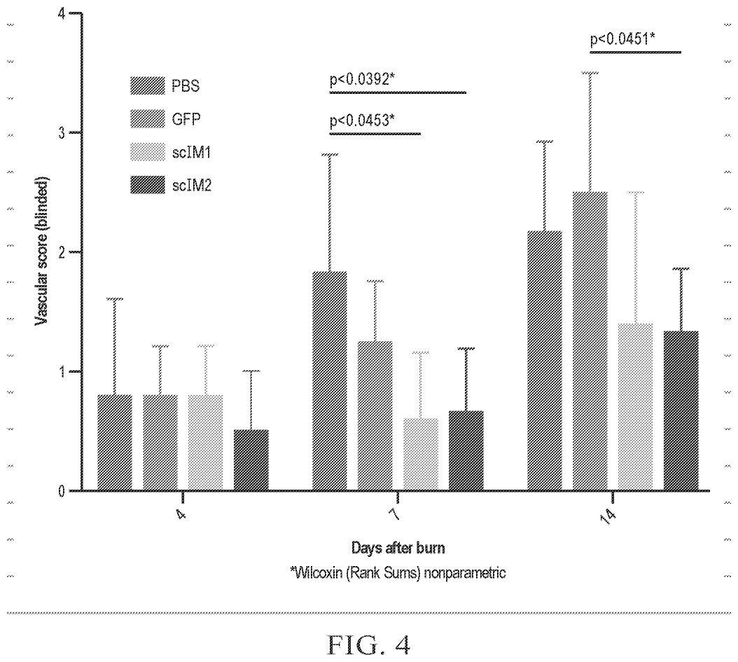

FIG. 4 is a graph that shows the vascular score for the prevention of corneal neovascularization post-NaOH corneal burn when treated with phosphate buffered saline (PBS), AAV-green fluorescence protein (GFP), AAV-scIM-1, or AAV-scIM-2, all injected subconjunctivally, at days 4, 7, and 14.

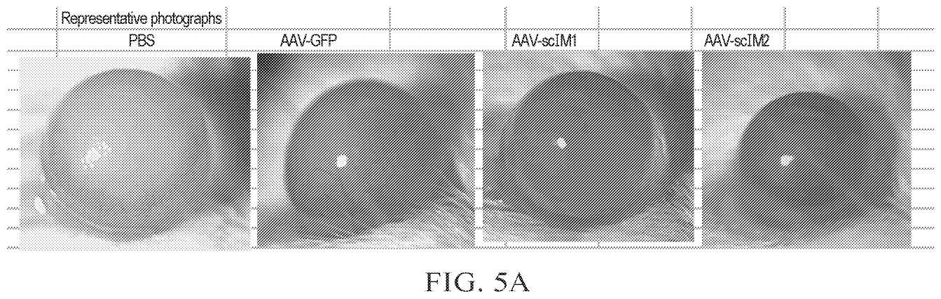

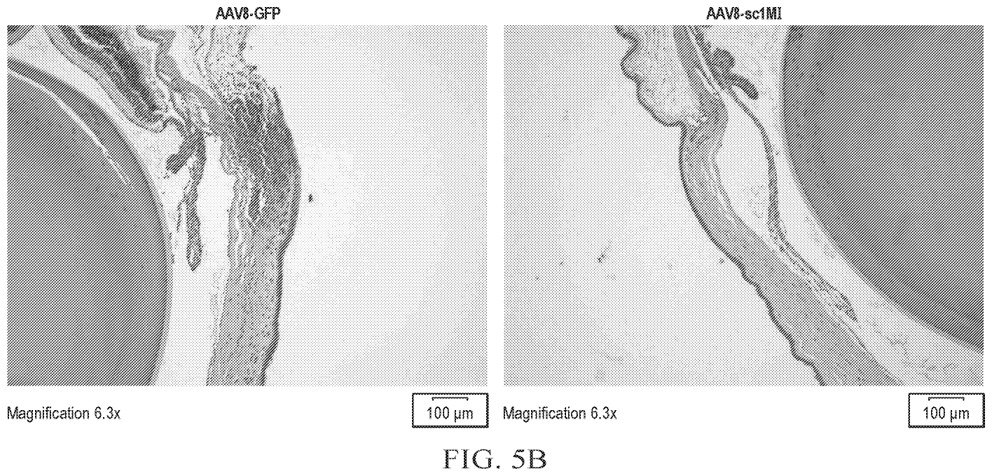

FIG. 5A shows that adeno-associated virus (serotype 8) scAAV8 encoding the single chain immune modulators (scIM) fusion proteins of the present invention (scIM-1 and scIM-2) prevented NaOH corneal burn induced neovascularization, the controls are animals treated with PBS and AAV8-GFP. FIG. 5B are histological cross-section of corneas treated with AAV8-GFP and AAV8-scIM1, demonstrating decreased burn-induced cellular infiltration of the cornea.

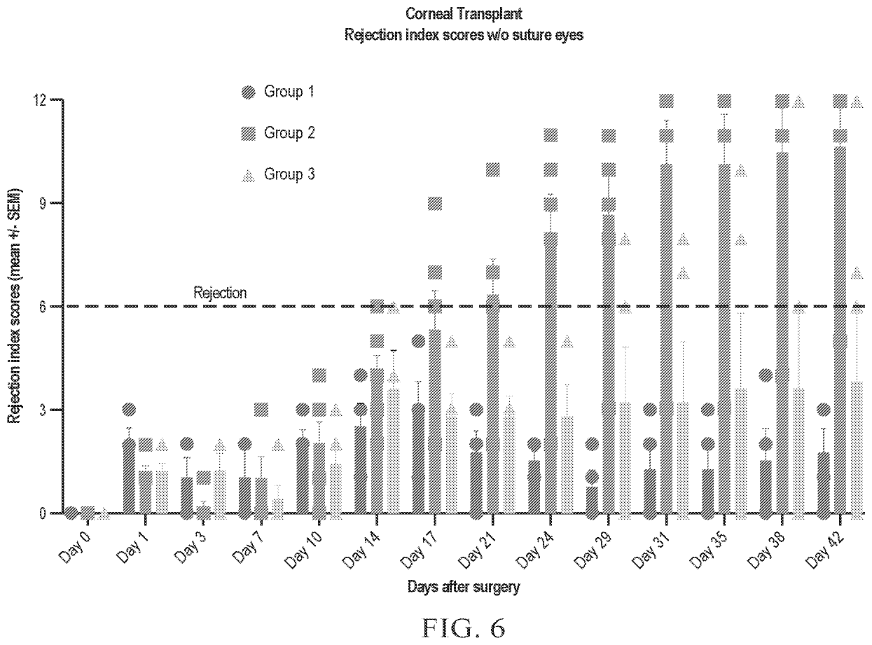

FIG. 6 shows the mean rejection index scores after full-thickness high-risk corneal transplantation of donor corneas incubated in AAV8-scIM1, AAV8-scIM2, or vehicle in rabbits. Rejection index (RI) score, a scale of 0 to 12, is based on the combined scores of 3 corneal graft indicators: clarity, edema, and neovascularization, each of which is scored on a scale of 0 to 4, with 0 being normal and 4 being of highest severity. A cornea with an RI score of 6 is considered rejected (dotted horizontal line). Corneas incubated with saline developed a mean RI of 6 between 17 and 21 days after surgery. Two of 6 eyes incubated in AAV8-scIM2 had RI scores of 6 by day 29 after surgery. The remaining corneas incubated in AA8-scIM2 and all corneas incubated in AAV8-scIM2 did not reject through day 42 after surgery.

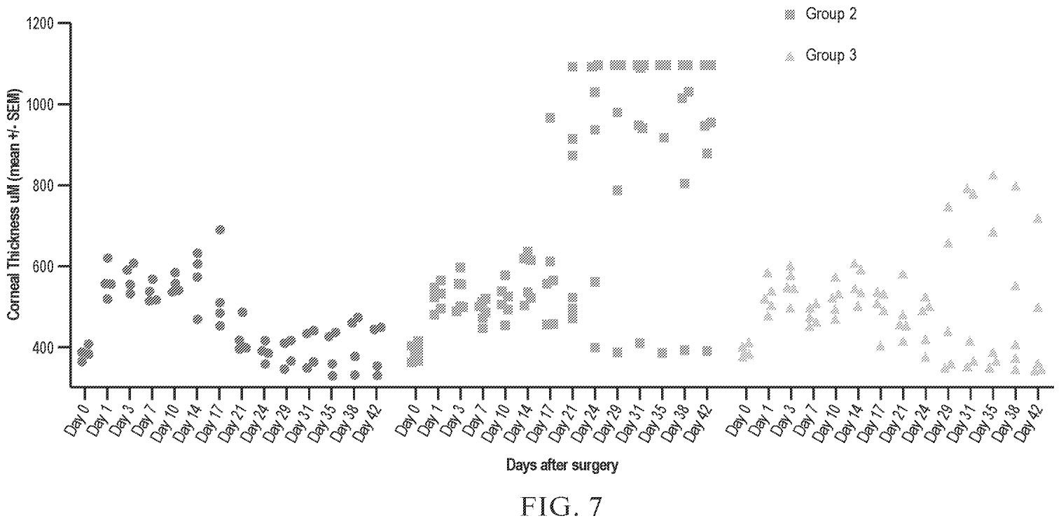

FIG. 7 shows individual central corneal thickness measurements after full-thickness high-risk corneal transplantation of donor corneas incubated in AAV8-scIM1, AAV8-scIM2, or vehicle in rabbits. A higher corneal thickness suggests corneal decompensation with inflammation. All but 2 corneas incubated with AAV8-scIM2 and all corneas incubated in AAV8-scIM1 had central corneal thickness measurements that returned to normal by day 21-24 days after surgery.

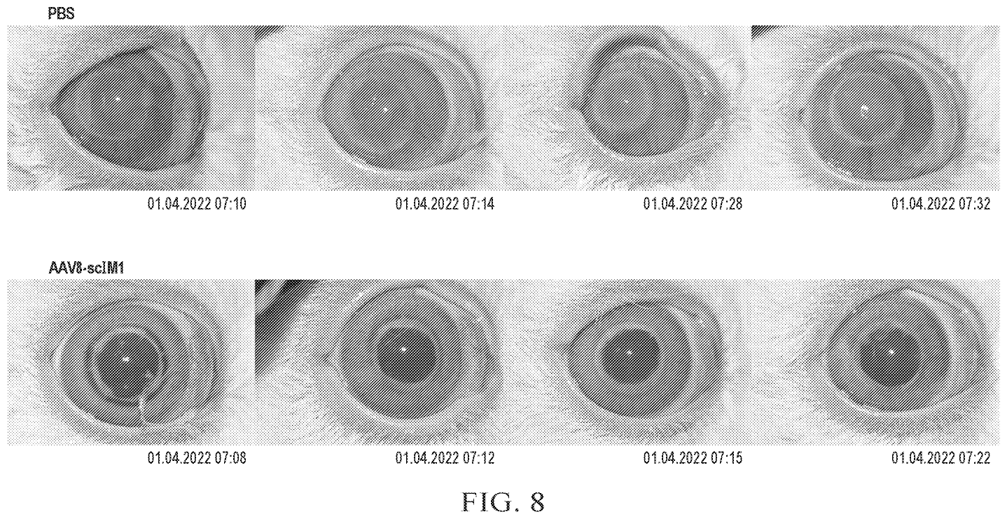

FIG. 8 shows photographs after full-thickness corneal transplantation of donor corneas incubated in AAV8-scIM1 or vehicle in rabbits. Healthy corneal grafts were incubated in vehicle or AAV8-scIM1 for 15 min prior to surgical engraftment into a high-risk corneal recipient bed. Representative photographs depict complete corneal graft rejection (PBS) or tolerance (AAV8-scIM1) at Day 38 post-engraftment.

As embodied and broadly described herein, an aspect of the present disclosure relates to a fusion protein comprising, consisting essentially of, or consisting of: a first HLA-G monomer and a second HLA-G monomer connected by a linker peptide expressed as a single chain. In one aspect, the linker peptide is selected from GG, KG, GK, GSG or GGG. In one aspect, the HLA-G is selected from HLA-G1, HLA-G2, HLA-G3, HLA-G4, HLA-G5, HLA-G6, or HLA-G7 or heteromonomers {G1,G1} {G1,G2} {G1,G3}{G1,G4} {G1,G5} {G1,G6} {G1,G7} {G2,G1} {G2,G2} {G2,G3} {G2,G4} {G2,G5} {G2,G6} {G2,G7}{G3,G1} {G3,G2} {G3,G3} {G3,G4} {G3,G5} {G3,G6} {G3,G7} {G4,G1} {G4,G2} {G4,G3} {G4,G4}{G4,G5} {G4,G6} {G4,G7} {G5,G1} {G5,G2} {G5,G3} {G5,G4} {G5,G5} {G5,G6} {G5,G7} {G6,G1}{G6,G2} {G6,G3} {G6,G4} {G6,G5} {G6,G6} {G6,G7} {G7,G1} {G7,G2} {G7,G3} {G7,G4} {G7,G5}{G7,G6} {G7,G7}. In another aspect, the fusion protein further comprises a providence virus globulin domain at an amino- or carboxy-terminus of the fusion protein. In another aspect, the nucleic acid that encodes the HLA-G has 75, 80, 98, 90, 95, 96, 97, 98, 99% or 100% sequence identity with the amino acid sequence of SEQ ID NO: 1 or 2. In another aspect, the HLA-G has amino acid sequence of SEQ ID NO: 3 or 4. In another aspect, the fusion protein is expressed by an expression vector in a bacteria, fungal, yeast, insect, plant, or mammalian cell. In another aspect, the fusion protein is expressed by a vector, RNA, or DNA delivered to a cell by a delivery vehicle or using a delivery method. In another aspect, the fusion protein is a soluble protein. In another aspect, the fusion protein has 75, 80, 98, 90, 95, 96, 97, 98, 99% or 100% sequence identity with the amino acid sequence of SEQ ID NOS: 6 or 8.

As embodied and broadly described herein, an aspect of the present disclosure relates to a method of making a fusion protein comprising, consisting essentially of, or consisting of: providing a nucleic acid vector that encodes a first HLA-G monomer, a linker sequence, and a second HLA-G monomer, wherein the fusion protein encoded by the nucleic acid forms a single-chain immune modulator protein that is an HLA-G dimer, wherein a transcript from the nucleic acid can be translated into the fusion protein. In one aspect, the linker peptide is selected from GG, KG, GK, GSG or GGG. In one aspect, the HLA-G is selected from HLA-G1, HLA-G2, HLA-G3, HLA-G4, HLA-G5, HLA-G6, or HLA-G7 or heteromonomers {G1,G1} {G1,G2} {G1,G3} {G1,G4} {G1,G5} {G1,G6} {G1,G7} {G2,G1} {G2,G2} {G2,G3} {G2,G4}{G2,G5} {G2,G6} {G2,G7} {G3,G1} {G3,G2} {G3,G3} {G3,G4} {G3,G5} {G3,G6} {G3,G7} {G4,G1}{G4,G2} {G4,G3} {G4,G4} {G4,G5} {G4,G6} {G4,G7} {G5,G1} {G5,G2} {G5,G3} {G5,G4} {G5,G5}{G5,G6} {G5,G7} {G6,G1} {G6,G2} {G6,G3} {G6,G4} {G6,G5} {G6,G6} {G6,G7} {G7,G1} {G7,G2}{G7,G3} {G7,G4} {G7,G5} {G7,G6} {G7,G7}. In another aspect, the fusion protein further comprises a providence virus globulin domain at an amino- or carboxy-terminus of the fusion protein. In another aspect, the nucleic acid that encodes the HLA-G has 75, 80, 98, 90, 95, 96, 97, 98, 99% or 100% sequence identity with the amino acid sequence of SEQ ID NO: 1 or 2. In another aspect, the HLA-G has 75, 80, 98, 90, 95, 96, 97, 98, 99% or 100% sequence identity with the amino acid sequence of amino acid sequence of SEQ ID NO: 3 or 4. In another aspect, the fusion protein is expressed by an expression vector in a bacteria, fungal, yeast, insect, plant, or mammalian cell. In another aspect, the fusion protein is expressed by a vector, RNA, or DNA delivered to a cell by a delivery vehicle or using a delivery method. In another aspect, the fusion protein is a soluble protein. In another aspect, the fusion protein has 75, 80, 98, 90, 95, 96, 97, 98, 99% or 100% sequence identity with the amino acid sequence of SEQ ID NOS: 6 or 8.

As embodied and broadly described herein, an aspect of the present disclosure relates to a single-chain immune modulator protein comprising, consisting essentially of, or consisting of: a nucleic acid encoding a first HLA-G monomer, a linker sequence, and a second HLA-G monomer, wherein the fusion protein encoded by the nucleic acid forms a single-chain immune modulator protein that resembles or mimics an HLA-G dimer. In one aspect, the HLA-G is selected from HLA-G1, HLA-G2, HLA-G3, HLA-G4, HLA-G5, HLA-G6, or HLA-G7 or heteromonomers {G1,G1} {G1,G2} {G1,G3} {G1,G4} {G1,G5}{G1,G6} {G1,G7} {G2,G1} {G2,G2} {G2,G3} {G2,G4} {G2,G5} {G2,G6} {G2,G7} {G3,G1} {G3,G2}{G3,G3} {G3,G4} {G3,G5} {G3,G6} {G3,G7} {G4,G1} {G4,G2} {G4,G3} {G4,G4} {G4,G5} {G4,G6}{G4,G7} {G5,G1} {G5,G2} {G5,G3} {G5,G4} {G5,G5} {G5,G6} {G5,G7} {G6,G1} {G6,G2} {G6,G3}{G6,G4} {G6,G5} {G6,G6} {G6,G7} {G7,G1} {G7,G2} {G7,G3} {G7,G4} {G7,G5} {G7,G6} {G7,G7}. In another aspect, the nucleic acid further comprises a providence virus globulin domain at an amino- or carboxy-terminus of the fusion protein, or a humanized providence virus globulin domain. In another aspect, the nucleic acid that encodes the HLA-G has SEQ ID NO:1 or 2. In another aspect, the HLA-G has amino acid sequence of SEQ ID NO:3 or 4. In another aspect, the fusion protein is expressed by an expression vector in a bacteria, fungal, yeast, insect, plant, or mammalian cell. In another aspect, the fusion protein is expressed by a vector, RNA, or DNA delivered to a cell by a delivery vehicle or using a delivery method. In another aspect, the nucleic acid expresses a fusion protein that is a soluble protein. In another aspect, the fusion protein has 75, 80, 98, 90, 95, 96, 97, 98, 99% or 100% sequence identity with the nucleic acid sequence of SEQ ID NOS: 5 or 7.

As embodied and broadly described herein, an aspect of the present disclosure relates to a method of preventing neovascularization comprising, consisting essentially of, or consisting of: providing an animal in need for the prevention of neovascularization with an effective amount of a fusion protein comprising a first HLA-G monomer and a second HLA-G monomer connected by a linker peptide expressed as a single chain. In one aspect, the linker peptide is selected from GG, KG, GK, GSG or GGG. In one aspect, the HLA-G is selected from HLA-G1, HLA-G2, HLA-G3, HLA-G4, HLA-G5, HLA-G6, or HLA-G7 or heteromonomers {G1,G1} {G1,G2} {G1,G3} {G1,G4} {G1,G5} {G1,G6} {G1,G7} {G2,G1}{G2,G2} {G2,G3} {G2,G4} {G2,G5} {G2,G6} {G2,G7} {G3,G1} {G3,G2} {G3,G3} {G3,G4} {G3,G5}{G3,G6} {G3,G7} {G4,G1} {G4,G2} {G4,G3} {G4,G4} {G4,G5} {G4,G6} {G4,G7} {G5,G1} {G5,G2}{G5,G3} {G5,G4} {G5,G5} {G5,G6} {G5,G7} {G6,G1} {G6,G2} {G6,G3} {G6,G4} {G6,G5} {G6,G6}{G6,G7} {G7,G1} {G7,G2} {G7,G3} {G7,G4} {G7,G5} {G7,G6} {G7,G7}. In another aspect, the fusion protein further comprises a providence virus globulin domain at an amino- or carboxy-terminus of the fusion protein. In another aspect, the nucleic acid that encodes the HLA-G has SEQ ID NO:1 or 2. In another aspect, the HLA-G has amino acid sequence of SEQ ID NO:3 or 4. In another aspect, the protein is expressed by an expression vector. In another aspect, the fusion protein is expressed by an expression vector in a bacteria, fungal, yeast, insect, plant, or mammalian cell. In another aspect, the fusion protein is expressed by a vector, RNA, or DNA delivered to a cell by a delivery vehicle or using a delivery method. In another aspect, the expression vector is a viral, bacteria, fungal, yeast, insect, plant, or mammalian expression vector. In another aspect, the fusion protein is a soluble protein. In another aspect, the fusion protein has 75, 80, 98, 90, 95, 96, 97, 98, 99% or 100% sequence identity with the amino acid sequence of SEQ ID NOS: 6 or 8.

As embodied and broadly described herein, an aspect of the present disclosure relates to a method of preventing or reducing neovascularization of the cornea comprising, consisting essentially of, or consisting of: providing an animal in need for the prevention of neovascularization of the cornea with an effective amount of a fusion protein comprising a first HLA-G monomer and a second HLA-G monomer connected by a linker peptide expressed as a single chain to prevent or reduce the neovascularization of the cornea.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

1. A fusion protein comprising:
a first HLA-G monomer and a second HLA-G monomer connected by a linker peptide expressed as a single chain.

2. The fusion protein of claim 1, wherein the linker peptide is selected from GG, KG, GK, GSG or GGG.

3. The fusion protein of claim 1, wherein the HLA-G is selected from HLA-G1, HLA-G2, HLA-G3, HLA-G4, HLA-G5, HLA-G6, or HLA-G7 or heteromonomers {G1,G1}{G1,G2} {G1,G3} {G1,G4} {G1,G5} {G1,G6} {G1,G7} {G2,G1} {G2,G2} {G2,G3} {G2,G4}{G2,G5} {G2,G6} {G2,G7} {G3,G1} {G3,G2} {G3,G3} {G3,G4} {G3,G5} {G3,G6} {G3,G7}{G4,G1} {G4,G2} {G4,G3} {G4,G4} {G4,G5} {G4,G6} {G4,G7} {G5,G1} {G5,G2} {G5,G3}{G5,G4} {G5,G5} {G5,G6} {G5,G7} {G6,G1} {G6,G2} {G6,G3} {G6,G4} {G6,G5} {G6,G6}{G6,G7} {G7,G1} {G7,G2} {G7,G3} {G7,G4} {G7,G5} {G7,G6} or {G7,G7}.

4. The fusion protein of claim 1, further comprising a providence virus globulin domain at an amino- or carboxy-terminus of the fusion protein.

5. The fusion protein of claim 1, wherein the nucleic acid that encodes the HLA-G has 75, 80, 98, 90, 95, 96, 97, 98, 99% or 100% sequence identity with the amino acid sequence of SEQ ID NO: 1 or 2;
the HLA-G has 75, 80, 98, 90, 95, 96, 97, 98, 99% or 100% sequence identity with the amino acid sequence of SEQ ID NO: 3 or 4; or
wherein the fusion protein has 75, 80, 98, 90, 95, 96, 97, 98, 99% or 100% sequence identity with the amino acid sequence of SEQ ID NOS: 6 or 8.

6. (canceled)

7. The fusion protein of claim 1, wherein at least one of:
the fusion protein is expressed by a vector, RNA, or DNA delivered to a cell by a delivery vehicle or using a delivery method;
the expression vector is a viral, bacteria, fungal, yeast, insect, plant, or mammalian expression vector; or
the fusion protein is a soluble protein.

8. (canceled)

9. (canceled)

10. (canceled)

11. A method of making a fusion protein comprising:
providing a nucleic acid vector that encodes a first HLA-G monomer, a linker sequence, and a second HLA-G monomer, wherein the fusion protein encoded by the nucleic acid forms a single-chain immune modulator protein that is an HLA-G dimer, wherein a transcript from the nucleic acid can be translated into the fusion protein.

12. The method of claim 11, wherein the linker peptide is selected from GG, KG, GK, GSG or GGG.

13. The method of claim 11, wherein the HLA-G is selected from HLA-G1, HLA-G2, HLA-G3, HLA-G4, HLA-G5, HLA-G6, or HLA-G7 or heteromonomers {G1,G1} {G1,G2}{G1,G3} {G1,G4} {G1,G5} {G1,G6} {G1,G7} {G2,G1} {G2,G2} {G2,G3} {G2,G4} {G2,G5}{G2,G6} {G2,G7} {G3,G1} {G3,G2} {G3,G3} {G3,G4} {G3,G5} {G3,G6} {G3,G7} {G4,G1}{G4,G2} {G4,G3} {G4,G4} {G4,G5} {G4,G6} {G4,G7} {G5,G1} {G5,G2} {G5,G3} {G5,G4}{G5,G5} {G5,G6} {G5,G7} {G6,G1} {G6,G2} {G6,G3} {G6,G4} {G6,G5} {G6,G6} {G6,G7}{G7,G1} {G7,G2} {G7,G3} {G7,G4} {G7,G5} {G7,G6} or {G7,G7}.

14. The method of claim 11, wherein the fusion protein further comprising a providence virus globulin domain at an amino- or carboxy-terminus of the fusion protein.

15. The method of claim 11, wherein the nucleic acid that encodes the HLA-G has 75, 80, 98, 90, 95, 96, 97, 98, 99% or 100% sequence identity with the amino acid sequence of SEQ ID NO: 1 or 2;
the HLA-G has 75, 80, 98, 90, 95, 96, 97, 98, 99% or 100% sequence identity with the amino acid sequence of SEQ ID NO: 3 or 4; or
the fusion protein has 75, 80, 98, 90, 95, 96, 97, 98, 99% or 100% sequence identity with the amino acid sequence of SEQ ID NOS: 6 or 8.

16. (canceled)

17. The method of claim 11, wherein at least one of:
the fusion protein is expressed by an expression vector in a bacteria, fungal, yeast, insect, plant, or mammalian cell;
the fusion protein is expressed by a vector, RNA, or DNA delivered to a cell by a delivery vehicle or using a delivery method; or
the fusion protein is a soluble protein.

18. (canceled)

19. (canceled)

20. (canceled)

21. A nucleic acid encoding a single-chain immune modulator protein fusion protein comprising:
a nucleic acid encoding a first HLA-G monomer, a linker sequence, and a second HLA-G monomer, wherein the fusion protein encoded by the nucleic acid forms a single-chain immune modulator protein that is an HLA-G dimer.

22. The nucleic acid of claim 21, wherein the HLA-G is selected from HLA-G1, HLA-G2, HLA-G3, HLA-G4, HLA-G5, HLA-G6, or HLA-G7 or heteromonomers {G1,G1}{G1,G2} {G1,G3} {G1,G4} {G1,G5} {G1,G6} {G1,G7} {G2,G1} {G2,G2} {G2,G3} {G2,G4}{G2,G5} {G2,G6} {G2,G7} {G3,G1} {G3,G2} {G3,G3} {G3,G4} {G3,G5} {G3,G6} {G3,G7}{G4,G1} {G4,G2} {G4,G3} {G4,G4} {G4,G5} {G4,G6} {G4,G7} {G5,G1} {G5,G2} {G5,G3}{G5,G4} {G5,G5} {G5,G6} {G5,G7} {G6,G1} {G6,G2} {G6,G3} {G6,G4} {G6,G5} {G6,G6}{G6,G7} {G7,G1} {G7,G2} {G7,G3} {G7,G4} {G7,G5} {G7,G6} or {G7,G7}.

23. The nucleic acid of claim 21, further comprising a providence virus globulin domain at an amino- or carboxy-terminus of the fusion protein.

24. The nucleic acid of claim 21, wherein the nucleic acid that encodes the HLA-G that has 75, 80, 98, 90, 95, 96, 97, 98, 99% or 100% sequence identity with the amino acid sequence of SEQ ID NO:1 or 2;
the HLA-G has 75, 80, 98, 90, 95, 96, 97, 98, 99% or 100% sequence identity with the amino acid sequence of SEQ ID NO:3 or 4; or
the fusion protein has 75, 80, 98, 90, 95, 96, 97, 98, 99% or 100% sequence identity with the nucleic acid sequence of SEQ ID NOS: 5 or 7.

25. (canceled)

26. The nucleic acid of claim 21, wherein the fusion protein is expressed by a vector, RNA, or DNA delivered to a cell by a delivery vehicle or using a delivery method, or the nucleic acid expresses a fusion protein that is a soluble protein.

27. (canceled)

28. (canceled)

29. A method of preventing neovascularization comprising:
providing an animal in need for the prevention of neovascularization with an effective amount of a fusion protein comprising a first HLA-G monomer and a second HLA-G monomer connected by a linker peptide expressed as a single chain.

30. The method of claim 29, wherein the linker peptide is selected from GG, KG, GK, GSG or GGG.

31. The method of claim 29, wherein the HLA-G is selected from HLA-G1, HLA-G2, HLA-G3, HLA-G4, HLA-G5, HLA-G6, or HLA-G7 or heteromonomers {G1,G1} {G1,G2}{G1,G3} {G1,G4} {G1,G5} {G1,G6} {G1,G7} {G2,G1} {G2,G2} {G2,G3} {G2,G4} {G2,G5}{G2,G6} {G2,G7} {G3,G1} {G3,G2} {G3,G3} {G3,G4} {G3,G5} {G3,G6} {G3,G7} {G4,G1}{G4,G2} {G4,G3} {G4,G4} {G4,G5} {G4,G6} {G4,G7} {G5,G1} {G5,G2} {G5,G3} {G5,G4}{G5,G5} {G5,G6} {G5,G7} {G6,G1} {G6,G2} {G6,G3} {G6,G4} {G6,G5} {G6,G6} {G6,G7}{G7,G1} {G7,G2} {G7,G3} {G7,G4} {G7,G5} {G7,G6} or {G7,G7}.

32. The method of claim 29, wherein the fusion protein further comprises a providence virus globulin domain at an amino- or carboxy-terminus of the fusion protein.

33. The method of claim 29, wherein the nucleic acid that encodes the HLA-G has 75, 80, 98, 90, 95, 96, 97, 98, 99% or 100% sequence identity with the amino acid sequence of SEQ ID NO:1 or 2;
wherein the HLA-G has 75, 80, 98, 90, 95, 96, 97, 98, 99% or 100% sequence identity with the amino acid sequence of SEQ ID NO:3 or 4;
the HLA-G is human HLA-G; or
the fusion protein is expressed by a vector, RNA, or DNA delivered to a cell by a delivery vehicle or using a delivery method.

34. (canceled)

35. (canceled)

36. (canceled)


SEQUENCE LISTING (9 sequences)

SEQ ID NO: 1 - DNA, 1578 bp, Homo sapiens
  misc_feature 1..1578: note = HLA-G, Accession No. NM_002127
  source 1..1578: mol_type = genomic DNA
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 2 - DNA, 1475 bp, Homo sapiens
  misc_feature 1..1475: note = HLA-G, Accession No. NM_001363567
  source 1..1475: mol_type = genomic DNA
agtgtggtactttgtcttgaggagatgtcctggactcacacggaaacttagggctacggaatgaagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 3 - protein, 338 aa, Homo sapiens
  REGION 1..338: note = misc_feature - HLA-G, Accession No. NP_002118.1
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 4 - protein, 342 aa, Homo sapiens
  REGION 1..342: note = misc_feature - HLA-G, Accession No. NP_001350496
KTPRMVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 5 - DNA, 1800 bp, synthetic construct
  misc_feature 1..1800: note = Synthetic: scIM1 nucleic acid sequence
  source 1..1800: mol_type = other DNA
atgaggtacttctccgccgccgtgtctcggccaggaagaggagagccacggttcatcgctatgggctatgtggacgatacccagttcgtgaggtttgacagcgattccgccggaccagagcggttctacgagaagcctcggatggagccaagagccccctgggtggagcaggagggacctgagtattgggaggaggagaccaggaacacaaaggcccacgcccagacagacaggatgaacctgcagaccctgcgcggctactataatcagtctgaggccagctcccacaccctgcagtggatgatcggatgcgacctgggaagcgatggcaggctgctgaggggatacgagcagtacgcctatgacggcaaggattatctggccctgaatgaggacctgcggtcctggacagcagcagataccgcagcccagatctctaagagaaagtgcgaggcagcaaacgtggcagagcagaggagagcatacctggagggaacctgcgtggagtggctgcaccggtatctggagaatggaccacctgcagagagattcagcgccggatccatgacagtgtctgccagcaacgccatctttcagccctccaataccgtggcccgcatcgtgtggtctatcacacctctgccagtggcaaccgtggccctgaccacaggaaccggcggcacaaacaatacctctggcaagttctttagcgtggagatcgacggcaacgtgaatagcgtgtggaccttcacagcaccagcatccatcctggcagagggagagcccttcgccgaggagggcgacaccacatccttttctatgaccacaatcacagccgataccgtggtgtactccgtgtctagctccctgacaggctctagcgtgatcgtgcggggagtgaccaagggatctggcgtgagcatcacaccagtgaccgtgggaatcgacacaggaggaggaatgaggtacttcagcgccgccgtgtcccggcctggaagaggagagccacgctttatcgctatgggctacgtcgatgacacccagttcgtgcggtttgattctggcagaatctttaagcaggtgccaggaagcgcccctaggatggagcctcgcgccccctgggtcgaacaggagggaccagagtactgggaggaggagacaagaaacaccaaagctcacgcccagacagatagaatgaacctgcagaccctgagaggctactataatcagtccgaggcctcctctcatactctgcagtggatgatcggctgtgacctgggctccgacggcaggctgctgagaggctatgaacagtatgcttacgatggcaaggactatctggccctgaatgaagacctgaggagctggaccgccgcagataccgcagcacagatctccaagaggaaatgtgaagccgccaacgtggccgagcagaggagggcatacctggagggaacatgcgtggaatggctgcataggtatctggagaatggcaaggagatgctgcagcgcgccgacccacccaagacacacgtgacccaccaccccgtgttcgattacgaggccaccctgaggtgctgggcactgggcttttatcctgccgagatcatcctgacatggcagagggacggagaggatcagacccaggacgtggagctggtggagacaagacctgccggcgatggcacctttcagaagtgggcagcagtggtggtgccaagcggagaggagcagaggtacacctgtcacgtgcagcacgagggactgccagagcctctgatgctgaggtggaagcagtga

SEQ ID NO: 6 - protein, 599 aa, synthetic construct
  REGION 1..599: note = Synthetic: scIM1 amino acid sequence
MRYFSAAVSRPGRGEPRFIAMGYVDDTQFVRFDSDSAGPERFYEKPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGPPAERFSAGSMTVSASNAIFQPSNTVARIVWSITPLPVATVALTTGTGGTNNTSGKFFSVEIDGNVNSVWTFTAPASILAEGEPFAEEGDTTSFSMTTITADTVVYSVSSSLTGSSVIVRGVTKGSGVSITPVTVGIDTGGGMRYFSAAVSRPGRGEPRFIAMGYVDDTQFVRFDSGRIFKQVPGSAPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQ

SEQ ID NO: 7 - DNA, 1857 bp, synthetic construct
  misc_feature 1..1857: note = Synthetic: scIM2 nucleic acid sequence
  source 1..1857: mol_type = other DNA
atggtcgtgatgaggtacttctccgccgccgtgagccggcctggaaggggagagcccaggttcatcgctatgggctacgtggacgacacccagttcgtgagattcgactccgacagcgccggaccaaagcctaggatggagccacgcgccccttgggtggagcaggagggccccgagtactgggaggaggagaccagaaacaccaaggcccacgcccagaccgacaggatgaacctgcagaccctgagaggctactacaaccagtccgaggccagctcccacaccctgcagtggatgatcggatgcgacctgggaagcgacggcaggctgctgagaggctacgagcagtacgcctacgacggcaaggactacctggccctgaacgaggacctgcggtcctggaccgcagcagacaccgcagcccagatcagcaagaggaagtgcgaggcagcaaacgtggcagagcagaggagggcctacctggagggcacctgcgtggagtggctgcacaggtacctggagaacggacctccagcagagagattctccgccggaagcatgaccgtgtccgccagcaacgccatcttccagccttccaacaccgtggccaggatcgtgtggagcatcacccctctgccagtggcaaccgtggccctgaccaccggaaccggaggaaccaacaacacctccggcaagttcttcagcgtggagatcgacggcaacgtgaactccgtgtggaccttcaccgcacctgcaagcatcctggcagagggagagccattcgccgaggagggcgacaccacctccttcagcatgaccaccatcaccgccgacaccgtggtgtactccgtgagctccagcctgaccggctccagcgtgatcgtgcggggagtgaccaagggatccggcgtgagcatcaccccagtgaccgtgggaatcgacaccggaggaggaatgagatacttctccgccgccgtgagccggccgggaaggggagagcctcgcttcatcgctatgggctatgtcgatgacacccagttcgtgcggttcgactccggcaggatcggaagcgcccctaggatggagcccagagccccctgggtcgaacaggaaggccccgagtattgggaggaggagaccaggaacaccaaggcacacgcacagaccgacagaatgaacctgcagaccctgcgcggatattataatcagtccgaggcctccagccatactctccagtggatgattggctgtgacctgggctccgatggcaggctgctcaggggatatgaacagtatgcttatgatggcaaggattacctggccctgaacgaagacctgaggtcctggactgccgcagacaccgcagcacagatcagcaagagaaaatgtgaagccgccaacgtggccgagcagaggagagcctacctggagggaacttgcgtggaatggctgcaccggtacctggagaacggcaaggagatgctgcagagggcagaccctcctaagacccacgtgacccaccaccccgtgttcgactacgaggccaccctgaggtgctgggcactgggcttctacccagccgagatcatcctgacctggcagagggacggagaggaccagacccaggacgtggagctggtggagaccagaccagcaggcgacggcaccttccagaagtgggcagcagtggtggtgcctagcggcgaggagcagcggtacacctgccacgtgcagcacgagggactgcctgagcctctgatgctgaggtggaagcagctcgagaggcctaataaagagctcagatgcatcgatcagagtgtgttggttttttgtgtgacgcgtaggaacccctagtga

SEQ ID NO: 8 - protein, 617 aa, synthetic construct
  REGION 1..617: note = Synthetic: scIM2 amino acid sequence
MVVMRYFSAAVSRPGRGEPRFIAMGYVDDTQFVRFDSDSAGPKPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGPPAERFSAGSMTVSASNAIFQPSNTVARIVWSITPLPVATVALTTGTGGTNNTSGKFFSVEIDGNVNSVWTFTAPASILAEGEPFAEEGDTTSFSMTTITADTVVYSVSSSLTGSSVIVRGVTKGSGVSITPVTVGIDTGGGMRYFSAAVSRPGRGEPRFIAMGYVDDTQFVRFDSGRIGSAPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQLERPNKELRCIDQSVLVFCVTRRNP

SEQ ID NO: 9 - protein, 136 aa, synthetic construct
  REGION 1..136: note = Synthetic: 2qqp
AERFSAGSMTVSASNAIFQPSNTVARIVWSITPLPVATVALTTGTGGTNNTSGKFFSVEIDGNVNSVWTFTAPASILAEGEPFAEEGDTTSFSMTTITADTVVYSVSSSLTGSSVIVRGVTKGSGVSITPVTVGID